(19) United States
(12) Patent Application Publication
(10) Pub. No.: US 2002/0142355 A1
(43) Pub. Date: Oct. 3, 2002

(54) Methods for the in vivo biotin labeling of polypeptides

(75) Inventors: Michael A. Barry, Pearland, TX (US); Michael B. Parrott, Houston, TX (US)

Correspondence Address: PILLSBURY WINTHROP, LLP, P.O. BOX 10500, MCLEAN, VA, 22102, US

(73) Assignee: Baylor College of Medicine

(21) Appl. No.: 09/987,485

(22) Filed: Nov. 14, 2001

Related U.S. Application Data
(60) Provisional application No. 60/247,965, filed on Nov. 14, 2000.

(51) Int. Cl. G01N 33/53; C12P 21/06; C07H 21/04; C12N 9/00; C12P 21/02; C12N 5/06; C12N 15/00; C12N 15/09; C12N 15/63; C12N 15/70; C12N 15/74; C12N 5/00; C12N 5/02

(52) U.S. Cl. 435/7.5; 435/183; 435/320.1; 435/69.1; 536/23.2; 435/325

(57) ABSTRACT

The present invention is directed to methods that can used for biotin labeling polypeptides in mammalian cells. The methods can be effectively used for cytoplasmic proteins, secreted proteins, and for proteins found on viral surfaces.

### CROSS REFERENCE TO RELATED APPLICATION

The present application claims the benefit of U.S. provisional application No. 60/247,965, filed on Nov. 14, 2000.

### FIELD OF THE INVENTION

The present invention is directed to methods for biotin labeling proteins in mammalian cells. The methods involve expressing the protein joined to a biotinylation-competent fusion partner.

### BACKGROUND OF THE INVENTION

Biotin is an agent commonly used in molecular biology to label biomolecules including drugs, carbohydrates, nucleic acids and proteins (see e.g., Green, Adv. Protein Res. 29:85-133 (1975)). In vitro biotin labeling procedures have been applied to a variety of drug targeting and viral gene therapy strategies (Ohno, et al., Biochem. Mol. Med. 58:227-233 (1996); Smith, et al., Proc. Nat'l Acad. Sci. USA 96:8855-8860 (1999)). However, in vitro procedures are not site specific, require prior purification of the substrate, and can lead to a labeled protein that is biologically inactive (Stolz, et al., FEBS Lett. 440:213-217 (1998)).

Recently, methods have been developed for using biotin to label specific proteins in vivo (Parrott, et al., Mol. Therapy 1:96-104 (2000); Cronan, J. Biol. Chem. 265:10327-10333 (1990); Murtif, et al., Proc. Nat'l Acad. Sci. USA 82:5617-5621 (1985); U.S. Pat. No. 5,932,433; Murtif, et al., J. Biol. Chem. 262:11813-11816 (1987); Shenoy, et al., FASEB J. 2:2505-2511 (1988); Shenoy, et al., J. Biol. Chem. 267:18407-18412 (1992)). This is usually accomplished by recombinantly joining DNA encoding the protein of interest to DNA encoding a peptide that serves as a substrate for a biotin-protein ligase. When the DNA is expressed in a host cell in the presence of the ligase enzyme and biotin, an amide bond is formed between the biotin carboxyl group and one or more specific lysine residues in the recombinant protein. A primary advantage of these in vivo procedures is that they tend to preserve the biological activity of the labeled proteins. Further advancements in in vivo biotinylation may make this technology the method of choice in gene therapy procedures of the future.

### SUMMARY OF THE INVENTION

The present invention is based upon the discovery of new methods for biotin-labeling cells in vivo. The methods take place in a transfected mammalian cell and have been found to produce labeled proteins which retain their biological activity.

In its first aspect, the invention is directed to a fusion protein that contains a biotinylation-competent protein or peptide selected from the group consisting of: pyruvate carboxylase, propionyl-CoA carboxylase, acetyl CoA carboxylase, methylcrotonyl-CoA carboxylase and a P. shermanii transcarboxylase domain (PSTCD) peptide. This is joined directly to the C-terminal end of a polypeptide of interest, preferably a polypeptide ordinarily encoded by a virus or mammalian cell. The term “joined directly” means that there are no intervening amino acids between the biotinylation-competent protein or peptide and the polypeptide of interest or in which a heterologous protein linker separates the two moieties. The term “PSTCD peptide” refers to either the full length PSTCD domain or to a portion of this domain which: (a) includes lysine 89 (the site of biotinylation, as shown in FIG. 1); (b) is at least 63 amino acids in length; and (c) undergoes biotinylation when expressed in a host cell. The full length PSTCD sequence is 122 amino acids in length and reads as follows: MKLKVTVNGTAYDVDVDVDKSHENPMGTILFGGGTGGAPAPAAGGAGAGKAGEG EIPAPLAGTVSKILVKEGDTVKAGQTVLVLEAMKMETEINAPTDGKVEKVLVKERD AVQGGQGLIKIG (SEQ ID NO: 1). In a preferred embodiment, a truncated form of the PSTCD domain is used that is 70 amino acids in length and has the sequence: EGEIPAPLAGTVSKILVKEGDTVKAGQTVLVLEAMKMETEINAPTDGKVEKVLVKER DAVQGGQGLIKIG (SEQ ID NO: 2).

The invention includes a polynucleotide vector that can be used for expressing the fusion protein described above. This vector should have a “coding region” consisting of nucleotides which code for the fusion protein and a promoter that is active in mammalian cells and operably linked to the coding region. The term “operably linked” refers to genetic elements that are joined in a manner that enables them to carry out their normal functions. For example, a gene is operably linked to a promoter when its transcription is under the control of the promoter and such transcription ultimately produces the polypeptide normally encoded by the gene. If the vector is used to transfect a mammalian cell with an endogenous pathway for biotinylating proteins, the biotinylation-competent portion of the fusion protein will become labeled. A preferred cell for carrying out this method of biotinylation is a CHO cell.

The general procedure of biotin-labeling described above includes several cases of special importance. In the first, the fusion protein consists of the biotinylation-competent protein or peptide (e.g., a PSTCD peptide) joined directly to a viral surface protein. One preferred viral surface protein is the fiber protein of adenovirus which has a region (the knob domain) that is responsible for the attachment of virus to cells during infection. Preferably, the biotinylation-competent protein or peptide used with the fiber protein is a PSTCD peptide between 63 and 70 amino acids in length, with the 70 residue peptide of SEQ ID NO:2 being most preferred.

The invention also encompasses a polynucleotide expression vector with a coding region in which nucleotides code for a fusion protein consisting of a viral surface protein and a biotinylation-competent protein or peptide. The coding region is operably linked to a promoter that is active in mammalian cells. This expression vector may be used in a method for biotin labeling a virus in which the virus is allowed to replicate in a mammalian host cell that expresses a biotin ligase, e.g., BirA or holocarboxylase synthetase, and that has been engineered to express the vector. For example, an adenovirus may be allowed to replicate in a mammalian cell that has been co-transfected with expression vectors encoding a fiber/PSTCD fusion protein and a prokaryotic biotin ligase (birA) from E. coli. Alternatively, transfection may take place in a cell line engineered to permanently and constituitively express BirA or that expresses its own endogenous biotin ligase (e.g., holocarboxylase synthetase).

A second labeling procedure of special importance involves the direct joining of a biotinylation-competent protein or peptide to the C-terminal or N-terminal end of a polypeptide having a leader sequence promoting its secretion from a mammalian cell. In a preferred embodiment, the biotinylation-competent protein or peptide is a PSTCD peptide corresponding to SEQ ID NO:2. Expression vectors may be made in which there is a coding region consisting of nucleotides encoding the fusion protein and an operably linked promoter.

The invention also encompasses a method for biotinylating a polypeptide secreted by a mammalian host cell. In this, the expression vector containing the polypeptide with a secretory leader sequence is used to transfect a cell engineered to express a second fusion protein consisting of a biotin ligase (e.g., BirA) directly linked to a leader sequence promoting secretion from the host cell. In one embodiment, the host cell is a CHO cell and the leader sequence linked to BirA is lgκ.

In another aspect, the invention is directed to a fusion protein consisting of a biotin acceptor peptide (BAP) joined directly to the C-terminal end of a polypeptide normally encoded by a virus or a mammalian cell. In one preferred embodiment, the biotin acceptor peptide has the sequence GLNDIFEAQKIEWH (SEQ ID NO:3). The invention includes a polynucleotide vector in which there is a coding region consisting of nucleotides encoding the fusion protein operably linked to a promoter active in mammalian cells.

The invention encompasses a method for biotinylating a cell surface polypeptide in vivo by expressing this vector in a host cell (e.g., a CHO cell) that has been engineered to also express a biotin ligase such as BirA. In an important embodiment, the PSTCD or BAP peptide is fused directly to a transmembrane protein such as the membrane-spanning domain of platelet-derived growth factor (PDGF). Biotin labeling of the surface protein and the cells may then be accomplished by expression of the vector in a mammalian host cell that has been engineered to express the cell surface protein along with an endogenous or exogenous biotin ligase enzyme.

Other mammalian or viral polypeptides may also be biotinylated using the vectors described above in a host cell expressing a biotin ligase. Preferably, the BAP peptide is joined directly to a VSV-G protein. Virus, particularly an enveloped virus, may then be biotin labeled by allowing the virus to replicate in a mammalian host cell that has been engineered to express both the BAP/VSG-G fusion protein and a biotin ligase. The most preferred viruses to use in this method are retroviruses, lentiviruses and herpesviruses.

The fusion proteins discussed above may be used to target a protein of interest to a cell in culture or in the body of a subject. This may be accomplished by: binding avidin to the surface of the cell; biotinylating the fusion protein; and then administering the biotinylated protein to either the medium surrounding the cell in culture or to the subject. In one preferred embodiment, the avidin is bound to the cell surface by attaching avidin to a ligand that binds to a receptor on the surface of the cell and then introducing the avidin/ligand molecule into either the cell culture medium or into the subject. This method will be of particular value when the protein of interest is on the surface of a virus and is used to target the virus to the cell.

### BRIEF DESCRIPTION OF THE DRAWINGS

FIG. 1: The figure shows a comparison of PSTCD, mouse pyruvate carboxylase and human acetyl-CoA carboxylase. The lysine biotinylated in each protein is indicated by the dark circle. N-terminal and C-terminal deletions are indicated by the arrows which point in the direction of the sequence remaining in the deletion construct.

### DETAILED DESCRIPTION OF THE INVENTION

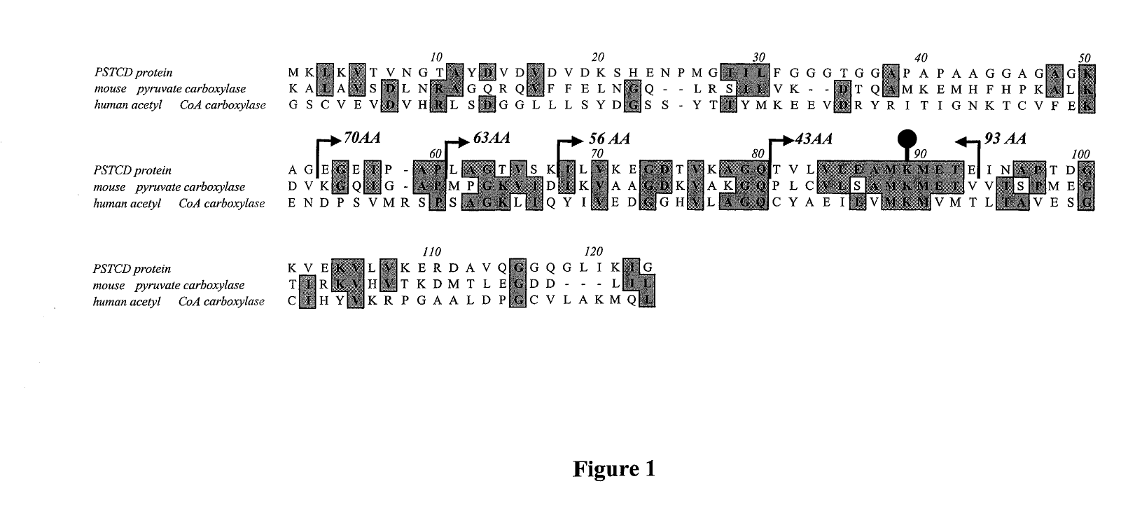

The present invention is directed to methods for biotin labeling proteins in a mammalian cell. In one approach, DNA encoding the protein of interest is ligated to a sequence encoding a biotinylation-competent polypeptide such as the PSTCD domain of E. coli. When expressed in a mammalian cell, this DNA produces a fusion protein which is biotinylated by an endogenous host cell enzyme. For example the lysine at position 89 of PSTCD as shown in FIG. 1 may be ligated. Using truncated forms of PSTCD and carrying out transfections in CHO cells, it was found that a substantial portion, up to 60 amino acids, of the PSTCD sequence could be eliminated without a complete loss of biotin labeling activity.

One of the main uses for the above procedure is to attach a biotin label to proteins on the surface of viral vectors used for delivering nucleic acids to cells in vivo. The biotin label provides a means for rapidly purifying virus (e.g., on a column of monomeric avidin), for attaching other compounds to the virus, for modifying the virus' ability to transduce cells in vivo and ex vivo, and, possibly, for directing the virus to specific avidin-tagged sites in a patient's body. One important virus that has been used as an in vivo vector is adenovirus. It has been found that the fiber protein on the surface of this virus may be successfully labeled by fusing it to a PSTCD peptide. In order to maintain the full activity of the virus, the PSTCD peptide should be no longer than 70 amino acids in length (e.g., SEQ ID NO:2).

A special problem arises when the protein being labeled is secreted from cells or is targeted to the cell surface. Because of compartmentalization, the endogenous enzyme responsible for transferring biotin to the expressed fusion protein may not be available. Thus, for secreted proteins, host cells should be engineered to express a biotin ligase, e.g., BirA or holocarboxylase synthetase, which has been modified by the attachment of a secretory leader sequence. For example, host cells may be co-transfected with a mammalian expression vector which has the birA gene immediately downstream from a lgκ leader sequence. Under these conditions, the BirA enzyme should co-compartmentalize with the secreted protein and be available to carry out labeling. Using the secreted human alpha-1-antitrypsin protein as a model, it was determined that successful labeling could be carried out using this procedure.

An alternative method of biotin-labeling proteins is similar to the one described above but utilizes a biotin acceptor peptide as the biotinylation-competent protein or peptide. Peptides suitable for this procedure have been described in the art (see e.g., Cronan, J. Biol. Chem. 265:10327-10333 (1990); Beckett, et al., Protein Sci. 8:921-929 (1999); Shatz, Biotechnology 11:1138-1143 (1993)). When this procedure is used, it is preferred that mammalian host cells be engineered to express a biotin ligase such as BirA. The procedure may be successfully used to label the VSV-G protein. When this is done in the presence of replicating retrovirus, biotin-labeled progeny virus are obtained which maintain a fully active VSV-G component or a VSV-G component that can be used as a scaffold for virus purification or the addition of exogenous substances.

Procedures for constructing DNA molecules with appropriately arranged elements are well known in the art and plasmids for expressing the fusion proteins described above can be made using standard techniques in molecular biology (see e.g. Sambrook, et al., Molecular Cloning: A Laboratory Manual, 2^{nd} ed., Cold Spring Harbor Press (1989)). Any type of promoter active in mammalian cells can be used in conjunction with the invention including those that are inducible, repressible or constitutive. Preferred mammalian promoters include that of the mouse metallothionein I gene (Hamer, et al., J. Mol. Appl. Gen. 1:273-288 (1982)); the immediate early and TK promoter of herpes virus (Yao, et al., J. Virol. 69:6249-6258 (1995)) McKnight, Cell 31:355-365 (1982)); the SV 40 early promoter (Benoist, et al., Nature 290:304-310 (1981)); and the human CMV promoter (Boshart, et al., Cell 41:521-530 (1985)). Full length or minimal promoters may be used and other regulatory elements may be included.

Any method for introducing expression vectors into cells is compatible with the invention including calcium phosphate precipitation, microinjection, electroporation, liposomal transfer, viral transfer or particle mediated gene transfer. Recombinant fusion protein that has been biotin labeled by cells can be purified on a column of monomeric avidin using procedures that are well known in the art. Such purification procedures may also be used to isolate virus that has incorporated a biotin-labeled protein on its surface. Mammalian host cells that may be used include, without limitation, NIH-3T cells, CHO cells, HeLa cells, LM (tk−) cells, as well as the cells of living animals and humans.

The proteins and virus labeled by the procedures described herein can be used in the same ways as their chemically labeled counterparts. However, because of the specificity inherent in enzymatic procedures, labeling is less destructive with respect to the biological functions of molecules. Thus, the proteins obtained using the present methods should generally retain their activity and virus should maintain its ability to deliver DNA to cells.

One particularly important application of the metabolic biotinylation systems described herein is for drug and gene therapy targeting. In these cases, mammalian targeting proteins or vectors could be produced already biotinylated from host mammalian cells. The ability to purify and release these targeting proteins using lower affinity monomeric avidin (Kd=10⁻⁷ M) combined with the ability to strongly attach bioactive drugs for targeting proteins via the affinity of tetrameric avidin (Kd=10⁻¹⁵ M) should make this system very attractive for targeting applications.

### EXAMPLES

### Example 1

Labeling of Cytoplasmic Proteins Using Endogenous Biotin Ligase Enzymes

Mammalian vectors were constructed to express a bacterially biotinylated carboxylase domain (PSTCD) fused to the C-terminus of enhanced green fluorescent protein (EGFP) and an EGFP-KNOB adenovirus type 5 fusion protein. These expression vectors were used to transfect CHO cells. After 24 hours, cells were lysed and the lysate run on 7.5% tricine-SDS PAGE gels. EGFP fusions and biotinylated proteins were detected with monoclonal anti-GFP and avidin-HRP. The Western blots demonstrate that the endogenous biotin ligase enzymes of mammalian cells can biotinylate the exogenous biotin-acceptor domains. This approach has been demonstrated both in cell culture and in living mice demonstrating the application in mammalian cells in or out of intact organisms.

Sequential truncations of PSTCD linked to the C-terminus of EGFP and the knob protein were made by long PCR mutagenesis. The full length PSTCD peptide and the truncated versions examined are shown in FIG. 1. After transfection and cell lysis, Western blots were performed and bands were visualized using antibody to EGFP and avidin-HRP. The results suggest that truncated forms of PSTCD that include lysine 89 must be at least 63 amino acids in length to undergo biotinylation.

### Example 2

Labeling of Cytoplasmic Proteins Using Exogenous Biotin Ligase Enzymes and Biotin Acceptor Peptides

Using transiently transfected CHO cells as described above, experiments were performed demonstrating that BirA the bacterial biotin ligase can be expressed in mammalian cells as a fusion to the C-terminus of EGFP and still retain its function as a biotin ligase. When co-transfected with constructs expressing GFP or the knob domain of the adenovirus fiber protein fused with a biotin acceptor peptide (BAP) at its C-terminus, it was found that site specific biotinylation occurred. The BAP sequence used in these experiments was GLNDIFEAQKIEWH (SEQ ID NO:3) having an 11 amino acid c-myc tag linked at its N-terminal end (see Beckett, et al., Prot. Sci. 8:921 (1999)). Thus, biotin acceptor peptides can be used in mammalian cells to achieve site specific biotinylation provided that an exogenous biotin kinase enzyme is coexpressed.

### Example 3

Labeling of Nuclear Proteins Using Endogenous Biotin Ligase Enzymes

Mammalian vectors were constructed to express the PSTCD biotin-acceptor domain fused to the C-terminus of the nuclear-localized adenovirus type 5 fiber protein. These expression vectors were used to transfect CHO cells and were analyzed by Western blot as described above. This work demonstrates that mammalian cells can biotinylate the PSTCD domain on a nuclear protein when fused to proteins in this manner.

### Example 4

Labeling of Nuclear Proteins Using Exogenous Biotin Ligase Enzymes

Mammalian vectors were constructed to express the BAP fused to the C-terminus of the nuclear adenovirus type 5 fiber protein. This expression vector and the GFP-BirA vector were used to transfect CHO cells and Western blot analysis was carried out as described above. This work demonstrates that mammalian cells can biotinylate the BAP domain on a nuclear protein when fused to proteins in this manner and when an exogenous biotin ligase enzyme is supplied. In addition, biotinylation of nuclear-localized fiber protein can be increased by expression of holocarboxylase synthase engineered with an SV40 nuclear-localization sequence.

### Example 5

Labeling of Secreted Proteins Using Endogenous and Exogenous Biotin Ligase Enzymes

In order to test for the ability to secrete endogenously biotinylated proteins from transfected cells, naturally secreted human alpha-1-antitrypsin (AAT) was fused to the N-terminus of PSTCD or the BAP expression constructs. TCA precipitation of tissue culture media after transfection revealed that, although there was an abundant presence of the fusion protein secreted into the medium (as detected by anti-AAT antibodies) none of the protein appeared to biotinylated, i.e., no bands were seen in Western blots probed with avidin-HRP.

In an attempt to establish a method for labeling secreted proteins from transfected cells, the birA gene (the prokaryotic biotin ligase) from E. coli was cloned into a mammalian expression vector behind the immunoglobulin secretory leader. The co-transfection of the secreted form of BirA reestablished biotinylation of the secreted proteins C-terminally tagged with the PSTCD domain or the BAP peptide. Similarly, co-secretion of holocarboxylase synthase engineered with a secretory leader mediates biotinylation of secreted proteins.

### Example 6

Labeling of Cell Surface Proteins

In order to test for the ability to express biotinylated proteins on the cell surface of transfected cells, the PSTCD or the BAP biotin acceptors were inserted between a secretory leader and the PDGF transmembrane domain or the vesicular stomatitis virus glycoprotein (VSV-g) in mammalian expression constructs. These constructs were transfected into mammalian cells with and without secreted BirA. Western blot, immunofluorescence, and flow cytometry analysis demonstrated the production of cell surface biotinylated proteins by the PSTCD-PDGF constructs in the absence of BirA demonstrating that the endogenous biotin ligases can biotinylate cell surface proteins. Addition of secreted BirA increased biotinylation of the cell-surface-targeted PSTCD domain. Analysis of the cell surface BAP expression revealed biotinylation of the cell surface proteins only in the presence of secreted-BirA. These observations demonstrate the ability to biotinylate cell surface proteins by the addition of large biotin acceptor domains like PSTCD or small biotin acceptor peptides like BAP using endogenous or exogenous biotin ligase enzymes. These observations also demonstrate the use of this approach to label the surface of mammalian cells with biotin for cell purification and the addition of exogenous moieties to the cells.

### Example 7

Labeling of Viral Proteins

Adenovirus type 5 is a non-enveloped virus that attaches to the CAR receptor (Coxsackie adenovirus receptor) on cells via the knob domain of its fiber protein. Fiber forms a trimeric structure on the capsid of the adenovirus. Recombinant or mutated fibers which do not trimerize generally will not propagate to significant viral titer do to an inability to form virus.

Experiments were conducted to determine whether a fusion between PSTCD and the adenoviral protein would be biotinylated and whether such labeling would interfere with trimer formation. Cells were transfected with a fusion protein consisting of fiber joined to either full length PSTCD or truncated versions of this domain. Western blots revealed that both the full length and truncated versions were labeled but that trimer formation did not occur with the full length form of the peptide. However, both substantial labeling and trimer formation was found to occur when a 70 amino acid form of PSTCD (SEQ ID NO:2) was used. This work demonstrates the ability to produce functional biotinylated viral proteins in the cytoplasm and nucleus of mammalian cells.

To further expand this demonstration, the GLNDIFEAQKIEWH (SEQ ID NO:3) BAP domain was inserted into a retroviral envelope protein. Retroviruses are enveloped viruses which bud from a cell surface after being replicated in the cytoplasm. As they bud, they carry the transmembrane envelope protein of the virus with them in addition to other membrane-associated proteins. Frequently, retroviral envelopes from different viruses are used to pseudotype viral vectors by simply co-transfecting the envelope protein of the pseudotyping virus with the vector of interest. In particular, retroviral vectors have been retargeted using bifunctional reagents to cap the envelope protein (Boerger, et al., Proc. Nat'l Acad. Sci. USA 96:9867). One of the most commonly used proteins for pseudotyping is the VSV-G protein.

Experiments were carried out in which VSVG-BAP constructs were transfected into cells together with DNA directing the expression of BirA. Results from Western blots indicated that VSV-G underwent extensive biotin labeling. It was also found that the labeled VSV-G retained its ability to mediate membrane fusion. Western blot analysis demonstrated the production of biotinylated VSV-g proteins on the cell surface of the transfected cells. This work demonstrates the ability of this approach to produce biotinylated proteins from enveloped and non-enveloped viruses.

### Example 8

Production of Biotinylated Viruses

Adenovirus fiber with the c-terminal 70 amino acid PSTCD domain described above was used to replace the wild-type fiber protein in an adenovirus production vector genome that expresses the luciferase or GFP reporter proteins. The resulting vector was transfected into 293 adenovirus helper cells where it subsequently formed viral plaques indicative of the production of replication competent adenovirus. Plaques were picked and expanded for a large scale adenovirus production which result in the production of approximately 10¹² virus particles per ml of vector comparable to parallel production of wild-type adenoviral vectors. This demonstrates that the addition of the PSTCD is compatible with production of this complex virus and allows production of normal amounts of the virus.

Western blot analysis of this virus purified twice on CsCl gradients demonstrated that the fiber proteins on the virus were biotinylated and thus the virus was biotinylated. Functional testing of the virus demonstrated that it retained the ability to transduce normal cells indicating that the addition of the PSTCD and its biotinylation did not disturb viral function. Therefore, this technology is able to production functional biotinylated viruses.

### Example 9

Retargeting of Biotinylated Viruses

One application area of the biotin technology discussed herein is for targeting proteins or viruses to specific cells for therapeutic applications. To demonstrate the functionality of this approach, the biotinylated adenoviral vectors described above were tested for the ability to re-direct their cell interactions by addition of avidin plus or minus a heterologous cell-binding ligand.

To test the functionality of the system, K562 cells (CD5 minus, CD59 positive, CD71 positive) were used as target cells and tetrameric avidin with biotinylated antibody against CD5, CD59, or CD71 was added. In this case, addition of antibodies against CD59 or CD71 resulted in 10 to 20-fold increased transduction of K562 over virus without avidin, whereas antibody against CD5 failed to increase transduction relative to controls. This work demonstrates proof of principle for the use of biotinylated viruses or proteins combined with heterologous biotinylated proteins via tetrameric avidin linkage as a method to re-target proteins or viruses to cells or proteins. It also provides proof of principle for the use of biotinylated proteins or viruses as core reagents to which any biotinylated moiety (drug, protein, carbohydrate, chemical, etc.) can be added to modify cell targeting or protein or virus function. This work further demonstrates the proof of principle for the use of biotinylated cell-specific ligands to mediate cell-target therapeutics when combined with biotinylated viruses or other agents.

One extended use of the technology has been demonstrated in which the biotinylated adenovirus is re-targeted to cells without a biotinylated ligand. In this case, the surface proteins of target cells (e.g., HeLa or K562 cells) were first chemically biotinylated with NHS-biotin, the cells were then coated with tetrameric avidin, and the biotinyated adenovirus added to the system. By this process, essentially many or all cell surface proteins are converted into a cognate receptor for the biotinylated vector and avidin. This approach allows increased transduction of cells that are normally permissive for the virus (e.g. HeLa cells) as well as greater than 10-fold increased transduction on cells without one or the other normal receptor for the virus (e.g., K562 cells). This work demonstrates the ability to combine chemical biotinylation of target cells to increase gene delivery with biotinylated adenovirus vectors. This provides proof of principle for increasing transduction or therapeutic delivery of biotinylated vectors or proteins in the absence of a cell-targeting ligand.

### Example 10

Metabolic Biotinylation of Secreted and Cell Surface Mammalian Proteins

A. Materials and Methods

Materials

Plasmid DNA purification columns were purchased from Qiagen (Chatsworth, Calif.). The PinPoint-Xa2 plasmid and Soft-Link Soft Release Avidin Resin were purchased from Promega (Madison, Wis.). pEGFP plasmids and anti-EGFP antibodies were purchased from Clontech (Palo Alto, Calif.). All oligonucleotides were synthesized by and purchased from Operon Technologies (Alameda, Calif.). Avidin-HRP was purchased from Vector Laboratories (Burlingame, Calif.). Secondary antibodies and SuperSignal chemiluminescent substrate were purchased from Pierce Chemical (Rockford, Ill.). RPMI-1640, antibiotic-antimycotic, and lipofectamine were purchased from Gibco-BRL (Gaithersburg, Md.). All other reagents were purchased from Sigma (St. Louis, Mo.), Fisher Scientific (Pittsburgh, Pa.), or VWR Scientific (Houston, Tex.).

Cell lines

Chinese hamster ovary (CHO) cells were maintained in RPMI-1640 media containing 10% fetal bovine serum (FBS) and antibiotic/antimycotic.

Plasmid constructs

pEGFP-C3 (Clontech) was used as the plasmid backbone for fusion of the PSTCD domain to the C-terminus of GFP in order to create the pEGFP-PSTCD construct as previously described (Parrott, et al., Mol. Ther. 1:96-104 (2000)). For the construction of the pSecAAT-PSTCD plasmid, the pSeci-AAT vector (whose construction was described previously (Wu, et al., Mol. Ther. 2:288-297 (2000)) was digested with NheI and ApaI allowing for the isolation by gel electrophoresis of the 1.3 kb fragment containing the secretory leader and full sequence of human α1-antitrypsin (SecAAT). SecAAT was then ligated into the pEGFP-PSTCD vector digested with NheI and ApaI in order to remove the intervening GFP sequence and replace it with the SecAAT sequence. The plasmid pSecAAT-GFP-PSTCD was created by cloning the AgeI/DraIII fragment from pEGFP-PSTCD into the same restriction sites present in the pEGFP-N1-SecAAT plasmid (also previously described (Wu, et al., Mol. Ther. 2:288-297 (2000)) thus creating a fusion of AAT at the N-terminus, GFP as the middle domain, and the PSTCD at the C-terminus. For construction of pSec-BirA, total genomic DNA was extracted from XL-1 Blue E. coli and the birA gene was amplified by PCR using the following oligonucleotides:

| | |
|---|---|
| 5′-CTCGGGCCCAGAATTCTCACCATGAAGGATAACACCGTGCCACTG-3′ | (SEQ ID NO: 4); and |
| 5′-CTCTCTAGAGCCTTTTTCTGCACTACGCAGGGATAT-TTC-3′ | (SEQ ID NO: 5). |

This amplified product was gel purified, digested with EcoR1 and XbaI, and ligated into the pEGFP-C3 plasmid digested with EcoR1 and XbaI (purified from a dam-host in order to allow for cleavage at the XbaI site) to create pEGFP-BirA. The birA gene was then subcloned by digestion of pEGFP-BirA with BglII and XbaI for insertion into pSec-TagA (Invitrogen) digested with BamH1 and XbaI in order to place the IgK secretion signal 5′ to birA. The pSecAAT-PSTCD-VSV-G construct was made by cloning the PSTCD into the pEGFP-N1 backbone 3′ to the SecAAT secretion leader and 5′ to the G-glycoprotein of vesicular stomatitis virus (VSV-G) which had been previously inserted into the plasmid backbone. Additionally, the PSTCD sequence was cloned into the pHook-2 plasmid (Invitrogen) in order to express the PSTCD as a cell surface membrane protein fused to the platelet derived growth factor receptor transmembrane domain (PDGFR(tm)). All constructs were sequenced to verify that the correct junctions were formed during each plasmid construction and the birA portion of pSecBirA was completely sequenced.

Cell transfection

Cells were freshly passed to 35 mm 6-well tissue culture dishes at a concentration of 7.5×10⁵ cells per well. Transfections were performed 16-20 h following seeding of the wells when the cells reached 60-80% confluence. Transfections were performed with 1 μg of plasmid DNA using LipofectAmine PLUS according to the manufacturer's recommendations. All transfections were supplemented with biotin to produce a final media concentration of 100 μM. Twenty-four hours following transfection, the cells were visualized on a Nikon E400 fluorescence microscope to confirm expression of the GFP fusion proteins prior to processing for Western blot analysis or protein purification.

SDS-PAGE and Western blot analysis

Cells from tissue culture transfection were drained of media, scraped from their plates and resuspended in 500 μL of PBS (137 mM NaCl, 2.7 mM KCl, 8.2 mM Na₂HPO₄, 1.5 mM KH₂PO₄, pH 7.4). The cells were then pelleted by centrifugation at 300 g for 10 min at 4° C. and resuspended in 50 μL of PBS and 50 μL of SDS-PAGE loading buffer. The samples were briefly sonicated on ice before being heated to 95° C. for 5 min. Ten microliters of the total sample was then loaded onto 7.5% Tricine-SDS-PAGE gels and electrophoresed at 100V for 100 min. The gels were then either stained with Coomassie blue to visualize total protein or were transferred onto nitrocellulose membranes by using a Bio-Rad Trans-Blot Semi-Dry Transfer Cell (Hercules, Calif.). The membranes were blocked in 5% non-fat milk-TBST (10 mM Tris, 150 mM NaCl, 0.5% Tween-20, pH 8.0) for 2 h at room temperature or overnight at 4° C. Blots were incubated at room temperature with avidin-HRP (diluted 1:2500 in TBST) or with anti-GFP peptide antibody (diluted 1:2500 in TBST) and then washed five times with TBST. Anti-GFP blots were then probed with an anti-rabbit-HRP conjugate (diluted 1:10000 in TBST) and, after a 1 h incubation, washed five more times with TBST. HRP labeled proteins were detected with SuperSignal chemiluminescent reagent on Hyperfilm ECL (Amersham Life Science; Buckinghamshire, ENG) with variable exposure times depending on the fluorescence of the individual blot.

Purification of biotinylated proteins

CHO cells were plated in 6 well plates as described above for transfection with the pSecAAT-PSTCD (1 μg/well) and pSecBirA (0.5 μg/well) constructs. Following the 3 h transfection time in serum-free media, the transfection media was removed and replaced with 1 mL per well of CHO-SFM supplemented with biotin (100 μM). After 24 h, 6 mL of supernatant was removed from the plates and cleared of any residual cells by two successive 10 min centrifugations of 600 g at 4° C. One milliliter was saved for analysis while the remaining 5 ml were processed further. The cleared supernatant was dialyzed against 500 mL of a biotin-free stabilizing buffer ((50 mM Tris-HCl, pH 7.5, 50 mM NaCl, 5% glycerol) with three buffer changes over a 48 h period. One milliliter of the dialyzed supernatant was saved for further analysis while the remainder was passed over a 500 μL column volume of a commercially available monomeric-avidin resin (Promega) equilibrated in stabilizing buffer. The column was then washed with three successive washes of 10 column volumes of stabilizing buffer and 1 mL of each wash was saved for analysis. Elution of the column was performed with 4 mL of stabilizing buffer supplemented with biotin to provide a final biotin concentration of 5 mM and 1 mL was saved for later analysis. Five hundred microliters of each 1 mL fraction were TCA precipitated and resuspended in 50 μL of protein loading buffer. Ten microliters of each sample was then loaded on SDS-PAGE gels as described above and parallel Western blots were examined with the use of Avidin-HRP and anti-hAAT as described above.

Immunohistochemistry

CHO cells were seeded and transfected as described above. Twenty-four hours following transfection, the media was removed from the cells, and they were washed 1× with PBS. The cells were fixed in 3.7% formaldehyde for 10 min and washed five times with 5% nonfat milk dissolved in PBS. The cells were blocked in 5% milk/PBS for 1 h at room temperature before the block was removed and replaced with avidin-HRP dissolved 1:300 in 5% milk/PBS for 1 h. The cells were then washed six times with PBS before being overlaid with Vectashield. The cells were then visualized by fluorescence microscopy as described above.

B. Results

Biotin Acceptor Domains are Not Biotinylated When Secreted from Mammalian Cells

Previous work has shown that the PSTCD domain could be used as a biotinylation tag for proteins expressed in the cytoplasm of mammalian cells. While this may be useful for some applications, it is also of interest to secrete biotinylated proteins into the media or onto the cell surface of mammalian cells. This would allow for additional applications of the avidin-biotin technology such as purification of biotinylated proteins from supernatants of continuously cultured cells, from transgenic animals, or for tagging the surface of transfected cells.

Previously, the GFP-PSTCD fusion protein has been shown to be fully biotinylated when targeted to the cytoplasm of both 293 (human embryonic kidney) and Chinese hamster ovary (CHO) cell lines. In order to investigate the biotinylation of secreted proteins, two additional mammalian expression plasmids were constructed to produce the fusion proteins. Human alpha-1 antitrypsin (hAAT) along with its secretory leader (secAAT) was cloned in frame 5′ to either green fluorescent protein (GFP) alone or 5′ to the GFP-PSTCD fusion. The N-terminal secAAT domain was expected to allow for secretion of the protein out of the cell via the endoplasmic reticulum along with its 3′ fusion protein.

The constructs were transfected into CHO cells using cationic liposomes and both media supernatants and whole cell lysates were collected 24 h following transfection. The lysates and supernatants were analyzed by Western blot using antibodies against either hAAT or GFP to identify the fusion proteins and with an avidin-HRP conjugate to determine if any of the fusion proteins were biotinylated. The GFP-PSTCD fusion protein appeared to be completely confined to the lysate and was not detected in the media by either the GFP antibody or the avidin-HRP conjugate as expected since there is no secretion signal 5′ to the fusion protein. The presence of the fusion protein was detected by avidin-HRP indicating that the protein was metabolically biotinylated by the mammalian cells.

Insertion of secAAT 5′ to GFP and to GFP-PSTCD did facilitate the secretion of these fusion proteins from the cells, although large amounts of each protein remain within the cell possibly due to steady state translocation of the protein through the secretory pathway. In addition, cells transfected with secAAT-GFP and secAAT-GFP-PSTCD actively secreted the fusion proteins into the media as demonstrated by their detection by the anti-hAAT antibodies. While secAAT-GFP-PSTCD appeared to be expressed and secreted from the cells as demonstrated by AAT antibody detection, the same proteins were not observed with avidin-HRP indicating the fusion proteins were not biotinylated by the mammalian cells. One hypothesis to explain these observations is that routing proteins through the secretion pathway may sequester the potential biotinylation site away from the endogenous mammalian cell biotin protein ligase activity necessary for them to be biotinylated. Although biotin ligase activity has previously been shown to be present in both the mitochondria and cytosol of mammalian cells, it has, to our knowledge, not been investigated as to whether biotin ligase is present within the secretion pathway of mammalian cells (Taroni, et al., J. Biol. Chem. 266:13267-13271 (1991)). Our data indicates that biotin ligase is not present in the secretory pathway which is not surprising since all of the endogenous carboxylases biotinylated by biotin ligase are present in either the cytosol or mitochondria and are never secreted. Given these observations, it appeared that normal mammalian cells could not metabolically biotinylate secreted proteins.

Cosecretion of the E. coli Biotin Ligase, BirA, from Mammalian Cells Enables Metabolic Biotinylation of Secreted Proteins

In order to investigate the possibility of biotinylating secreted proteins from mammalian cells, we reasoned that if the endogenous biotin ligase was not present in the secretory pathway, coexpression of a secreted biotin ligase might rescue this process. To test this hypothesis, we used BirA, the biotin protein ligase of E. coli, for this purpose, since BirA normally biotinylates the transcarboxylases present in E. coli and has also been used to biotinylate cytoplasmic recombinant proteins tagged with biotin acceptor peptides expressed in Spodoptera frugiperda cells. It was thus possible that BirA might also be able to biotinylate biotin acceptor protein domains fused to secreted proteins if BirA could be cosecreted with the fusion protein of interest.

To test this, the birA gene from E. coli was amplified from the bacterial genome and inserted it into a mammalian expression vector designed to target the protein to the endoplasmic reticulum by fusion to the IgK immunoglobulin secretory leader. This secreted BirA construct (pSecBirA) was cotransfected into CHO cells along with cytoplasmic and secretion targeted fusion proteins and the supernatants and total cell lysates were harvested as above for analysis by Western blot. The results indicated that the GFP-PSTCD construct was still biotinylated and detected almost exclusively in the total cell lysates. In contrast, the SecAAT-PSTCD and SecAAT-GFP-PSTCD constructs were secreted regardless of whether they were cotransfected with Sec-BirA since all four transfections appeared to yield readily detectable protein on the anti-hAAT Western blots.

None of the secreted proteins were biotinylated in the absence of exogenous BirA. In contrast, coexpression of secreted BirA rescued biotinylation of both the SecAAT-PSTCD and SecAAT-GFP-PSTCD proteins. The SecAAT-PSTCD and SecBirA cotransfected cells produced two biotinylated protein bands present in the total cell lysates. The smaller band represented a protein of approximately 59 kDa which is most likely SecAAT-PSTCD trapped in the cytoplasm or in the endoplasmic reticulum prior to glycosylation. The larger 66 kDa species was the same size as the secreted fusion protein and corresponds to the glycosylated form of the AAT fusion protein. The only form seen in the secreted protein was the larger 66 kDa form, confirming the necessity of routing an enzyme with biotin protein ligase activity to the endoplasmic reticulum in order to biotinylate secreted proteins. Thus, routing the BirA biotin ligase through the secretory pathway where the PSTCD fused proteins are sequestered rescues the ability to metabolically biotinylate these recombinant secreted proteins.

Biotinylated Proteins Secreted from Mammalian Cells Can Be Easily Purified from Mammalian Cell Supernatants

Being able to biotinylate secreted proteins from mammalian cells allows for the possibility of purifying recombinant proteins in a one-step fashion from mammalian cell supernatants. Usage of secreted biotin tagged protein purification has several advantages over other purification tag approaches. First, secreting proteins into the media allows for continuous culture of cells such that proteins can be harvested from cell supernatants without the necessity of lysing the cells to recover the protein of interest. This secretion provides a first level of purification which predominantly separates the protein of interest from the intracellular proteins and components of mammalian cells. An additional benefit of using the in vivo biotinylated proteins is that the avidin affinity columns used to purify biotinylated proteins have been shown to allow for purification of large amounts of protein from bacteria in essentially a one-step protocol (Parrott, et al., Mol. Ther. 1:96-104 (2000); Lesley, et al., J. Immunol. Methods 207:147-155 (1997)).

To investigate the purification of secreted biotinylated proteins from mammalian cells, CHO cells were transfected with the SecAAT-PSTCD and Sec-BirA constructs described above, and the supernatants were harvested from the cells after 24 h. The supernatants were then dialyzed in a standard stabilizing buffer in order to remove the excess biotin present in the media which had been added to increase the efficiency of the biotin ligation reaction (Parrott, et al., Mol. Ther. 1:96-104 (2000); Chapman-Smith,. Biochem J. 302: 881-887 (1994)). Following dialysis, the supernatant was passed over a monomeric avidin affinity column, washed with the stabilizing buffer, and eluted using 5 mM biotin. Samples from the various steps of purification were then analyzed by Western blots of SDS-PAGE gels which revealed that a significant portion of the biotinylated protein could be captured and eluted with biotin. Note, no pH changes, harsh salt conditions, or multiple columns were necessary to purify the protein. Thus, this protocol provides a simple one-step nondenaturing method of purifying proteins secreted from mammalian cells.

Cell Surface Biotinylation Can Be Achieved with PSTCD-Tagged Membrane Proteins Cosecreted with BirA

Having demonstrated the ability to biotinylate secreted proteins, we investigated if a similar system could be used to biotinylate cell-surface transmembrane proteins. Potential applications for biotinylation of cell-surface proteins include the ability to purify transfected cells from homogenous populations by monomeric avidin columns, magnetic beads, or FACS analysis which could provide for multiple applications of this technology. To test this, the PSTCD domain was cloned between the AAT secretory leader and the N-terminus of the G-glycoprotein of vesicular stomatitis virus (VSV-G). VSV-G is a transmembrane protein expressed on the cell surface of VSV infected cells. Currently, it is frequently used to pseudotype retroviral vectors because of its broad host range for human cells and the increased stability it imparts to retroviral preparations (Wang, et al., Gene 182:145-150 (1996); DePolo, et al., Mol. Ther. 2:218-222 (2000)).

The PSTCD-VSV-G fusion protein was tested by transfecting the plasmid into CHO cells in the absence or presence of secreted BirA. Western blots of the CHO cell lysates revealed that the VSV-G protein fused to PSTCD was biotinylated in a BirA dependent manner similar to that reported herein for secreted proteins. Surface expression of the biotinylated VSV-G was confirmed by flow cytometry using an avidin-FITC conjugate demonstrating the presence of the biotinylated protein on the cell surface.

While the biotinylation of secreted proteins appeared to be strictly BirA-dependent, the BirA-dependent biotinylation of cell-surface proteins appeared to be transmembrane domain-specific. The PSTCD fusion with VSV-G was biotinylated in a BirA-dependent fashion on the cell surface. In contrast, display of the PSTCD domain on the platelet-derived growth factor receptor transmembrane domain led to biotinylation of the domain regardless of whether secreted BirA was present or not. We hypothesize that the reason for this variability between the VSV-G and PDGFR linkages is the nature of the two transmembrane domains. The VSV-G protein is targeted to the cell membrane for the purpose of budding off with retroviral nucleocapsids as they are formed in the cytoplasm. In contrast, the PDGF receptor normally is turned over at the cell surface by endocytosis at a rapid rate. Thus, its ability to rapidly cycle on and off of the cell membrane may allow it to be exposed to the endogenous biotin ligase activity of mammalian cells.

The results demonstrate that cell-surface proteins can be metabolically biotinylated on mammalian cells and that this process may require cosecretion of a biotin ligase for some transmembrane proteins. The work further demonstrates the utility of the metabolic biotinylation technology for biotinylating cytoplasmic, nuclear, secreted, and cell surface proteins from mammalian cells. This technology therefore may allow for the ready tagging and purification of proteins, viruses, and whole mammalian cells using the potent avidin-biotin system.

C. Discussion

The strong noncovalent interaction between avidin and biotin allows for a variety of biological applications of these reagents. Here we have demonstrated an expansion of this technology for use in mammalian cells for the production of secreted and cell surface biotin labeled proteins. Through the use of a biotin acceptor tag derived from the 1.3S subunit of the P. shermanii transcarboxylase (PSTCD), it is possible to biotinylate proteins secreted from and expressed on the surface of mammalian cells fused with the PSTCD.

In order to accomplish biotinylation of secreted proteins and cell surface proteins, we investigated the use of BirA, a biotin protein ligase derived from E. coli. All eukaryotic and prokaryotic organisms have only one biotin ligase, which is necessary for the transfer of biotin onto the carboxylases and decarboxylases necessary for the normal metabolism of the organism. The carboxylases require biotin as a cofactor in order to facilitate carboxyl transfer from one organic molecule to another. In this work, we determined that the bacterial BirA could be expressed and function in the mammalian secretory pathway.

We found that the primary requirement for metabolic biotinylation of secreted proteins routed through the mammalian secretion pathway is that a biotin protein ligase must be cosecreted, since biotin ligase is not normally present in this cellular compartment. Our results demonstrate that cosecretion of BirA in mammalian cells with PSTCD-tagged proteins results in robust biotinylation of the secreted-tagged proteins and of cell membrane proteins into which the biotin acceptor domains have been incorporated.

Multiple applications of this technology exist and have been demonstrated here. One potentially powerful application is the ability to purify PSTCD-tagged proteins in a simple one-step nondenaturing protocol. Using the PSTCD tag, protein fusions can be easily purified from mammalian cell supernatants and concentrated on monomeric avidin columns with few manipulations. This approach has utility for the purification of secreted mammalian proteins that can only be correctly folded or post-translationally processed in mammalian cells. This application may also be useful for cell culture applications as well as for the purification of recombinant proteins from transgenic animals.

Another application of this technology is the membrane labeling of transfected cells for tagging or purification purposes using either monomeric avidin reagents, magnetic beads, or flow cytometry. Since no other biotinylated cell surface protein exists in nature, this cell surface label should be specific and have the advantage of compatibility with the diverse array of biotin-avidin reagents available. The observation that biotin-tagged VSV-G and PDGFR transmembrane domains behave differently with respect to biotinylation and cell sorting may point towards the application of these tagged molecules for the specific tracking of individual membrane proteins during their response to signaling or ligand interaction. The ability to biotinylate proteins on the cell surface also opens up the possibility of allowing for the external attachment of biotinylated or avidinated drugs, proteins, viruses, or tissue-engineering scaffolds to cells expressing biotinylated cell surface molecules. Thus, the use of avidin-biotin technology in mammalian cells appears as flexible and useful as the current widespread usage of the interaction in prokaryotic applications. In addition, this work demonstrates the unique applications of metabolic biotinylation to produce reagents that can only be derived and used in mammalian cells or in living mammals.

What is claimed is:

1. A fusion protein consisting essentially of:
a) a biotinylation-competent protein or peptide;
b) a polypeptide of interest;
and wherein said biotinylation-competent protein or peptide is joined directly to the N- or C-terminal end of said polypeptide of interest.

2. The fusion protein of claim 1, wherein said biotinylation-competent protein or peptide is selected from the group consisting of: pyruvate carboxylase; propionyl-CoA carboxylase; acetyl CoA carboxylase; methylcrotonyl-CoA carboxylase; and a PSTCD peptide wherein said PSTCD peptide consists of either the full length PSTCD domain as shown in SEQ ID NO:1 or a portion of the PSTCD domain which: (a) includes lysine 89; (b) is at least 63 amino acids in length; and (c) undergoes biotinylation when expressed in a host cell.

3. The fusion protein of claim 2, wherein said biotinylation-competent protein or peptide is a PSTCD peptide consisting of either the full length PSTCD domain as shown in SEQ ID NO:1 or a portion of the PSTCD domain which: (a) includes lysine 89; (b) is at least 63 amino acids in length; and (c) undergoes biotinylation when expressed in a host cell.

4. The fusion protein of claim 3, wherein said biotinylation-competent PSTCD peptide is 70 amino acids in length and has a sequence corresponding to that of SEQ ID NO:2.

5. A polynucleotide vector for expressing protein comprising:
a) a coding region consisting of nucleotides encoding the fusion protein of claim 1; and
b) a promoter active in mammalian cells and operably linked to said coding region.

6. A method for biotinylating a polypeptide of interest, comprising expressing the vector of claim 5 in a mammalian host cell in vivo or in vitro.

7. The method of claim 5, wherein said cell is a CHO cell in culture.

8. The fusion protein of claim 1, wherein said polypeptide of interest is a viral surface protein.

9. The fusion protein of claim 8, wherein said viral surface protein is the fiber protein of adenovirus and said biotinylation-competent protein or peptide is a PSTCD peptide is 70 amino acids in length and having a sequence corresponding to that of SEQ ID NO:2.

10. A polynucleotide vector for expressing protein comprising:
a) a coding region consisting of nucleotides encoding the fusion protein of claim 8; and
b) a promoter active in mammalian cells and operably linked to said coding region.

11. A method for biotin-labeling a virus, comprising replicating said virus in a mammalian host cell, wherein said host cell expresses a biotin ligase and has been engineered to express the vector of claim 10.

12. The method of claim 11, wherein said virus is a non-enveloped virus.

13. The method of claim 12, wherein said non-enveloped virus is an adenovirus.

14. The method of claim 11, wherein said host cell has been engineered to express BirA.

15. The fusion protein of claim 1, further comprising a leader sequence that promotes the secretion of said fusion protein from a mammalian host cell.

16. The fusion protein of claim 15, wherein said biotinylation-competent protein or peptide is joined directly to the C-terminal end of a polypeptide with a leader sequence, wherein said leader sequence promotes secretion from a mammalian host cell.

17. The fusion protein of claim 15, wherein said biotinylation-competent protein or peptide is a PSTCD peptide is 70 amino acids in length and having a sequence corresponding to that of SEQ ID NO:2.

18. A polynucleotide vector for expressing protein, comprising:
a) a coding region consisting of nucleotides encoding the fusion protein of claim 15; and
b) a promoter active in mammalian cells and operably linked to said coding region.

19. A method for biotinylating a polypeptide secreted by a mammalian host cell, comprising expressing the vector of claim 18 in said host cell, wherein said host cell has been engineered to express a distinct fusion protein consisting of a biotin ligase directly linked to a leader sequence that promotes secretion from said host cell.

20. The method of claim 19, wherein said biotin ligase is BirA.

21. The method of claim 20, wherein said host cell is a CHO cell and said leader sequence linked to BirA is the lgκ secretory leader.

22. A fusion protein consisting essentially of a biotin acceptor peptide (BAP) joined directly to the N- or C-terminal end of a polypeptide of interest.

23. The fusion protein of claim 22, wherein said biotin acceptor protein has the sequence of SEQ ID NO:3.

24. A polynucleotide vector for expressing protein, comprising:
a) a coding region consisting of nucleotides encoding the fusion protein of claim 22; and
b) a promoter active in mammalian cells and operably linked to said coding region.

25. A method for biotinylating a polypeptide of interest in vivo or in vitro, comprising expressing the vector of claim 22 in a mammalian host cell, wherein said host cell has been engineered to express a biotin ligase.

26. The method of claim 25, wherein said biotin ligase is BirA.

27. The fusion protein of claim 22, wherein said biotin acceptor protein is joined directly to the VSV-G protein.

28. The fusion protein of claim 27, wherein said biotin acceptor peptide has the sequence of SEQ ID NO:3.

29. A polynucleotide vector for expressing protein, comprising:
a) a coding region consisting of nucleotides encoding the fusion protein of claim 27; and
b) a promoter active in mammalian cells and operably linked to said coding region.

30. A method of biotin-labeling a virus, comprising: replicating said virus in a mammalian host cell, wherein said mammalian host expresses a biotin ligase and has been engineered to express the vector of claim 29.

31. The method of claim 30, wherein said virus is an enveloped virus.

32. The method of claim 31, wherein said enveloped virus is a retrovirus.

33. The method of claim 32, wherein said host cell has been engineered to express BirA.

34. A method of targeting a protein of interest to a cell in culture or in the body of a subject, comprising:
a) binding avidin to the surface of said cell;
b) biotinylating the fusion protein of claim 1, wherein said protein of interest protein of interest is joined to a biotinylation-competent protein or peptide;
c) administering the biotinylated protein of step c) to either to the medium surrounding said cell in culture or to said subject.

35. The method of claim 34, wherein said avidin is bound to the surface of said cell by a process comprising:
a) attaching avidin to a ligand that binds to a receptor located on the surface of said cell;
b) administering the avidin/ligand molecule of step a) either to the medium surrounding said cell in culture or to said subject.

36. The method of either claim 34 or claim 35, wherein said protein of interest is on the surface of a virus and is used to target said virus to said cell.


SEQUENCE LISTING (5 sequences)

SEQ ID NO: 1 - protein, 122 aa, Escherichia coli
MKLKVTVNGTAYDVDVDVDKSHENPMGTILFGGGTGGAPAPAAGGAGAGKAGEGEIPAPLAGTVSKILVKEGDTVKAGQTVLVLEAMKMETEINAPTDGKVEKVLVKERDAVQGGQGLIKIG

SEQ ID NO: 2 - protein, 70 aa, Escherichia coli
EGEIPAPLAGTVSKILVKEGDTVKAGQTVLVLEAMKMETEINAPTDGKVEKVLVKERDAVQGGQGLIKIG

SEQ ID NO: 3 - protein, 14 aa, Escherichia coli
GLNDIFEAQKIEWH

SEQ ID NO: 4 - DNA, 45 bp, Escherichia coli
ctcgggcccagaattctcaccatgaaggataacaccgtgccactg

SEQ ID NO: 5 - DNA, 39 bp, Escherichia coli
ctctctagagcctttttctgcactacgcagggatatttc